(19) United States
(12) Patent Application Publication
(10) Pub. No.: US 2025/0113872 A1
(43) Pub. Date: Apr. 10, 2025

(54) AEROSOL-GENERATING DEVICE WITH DISPLAY

(71) Applicant: Philip Morris Products S.A., Neuchatel (CH)

(72) Inventors: Enrico STURA, Palezieux-Village (CH); Mathias Torsten Rickard TINGSTRÖM, Morges (CH); Ezequiel Martin VALDEZ ROJAS, Pully (CH); Valerio OLIANA, Pully (CH)

(73) Assignee: Philip Morris Products S.A., Neuchatel (CH)

(21) Appl. No.: 18/572,413

(22) PCT Filed: Jun. 27, 2022
(86) PCT No.: PCT/EP2022/067609

§ 371 Date: Dec. 20, 2023

(30) Foreign Application Priority Data
Jun. 30, 2021 (EP) 21182965.0

(51) Int. Cl. A24F 40/60; A24F 40/20; A24F 40/40; A24F 40/50

(52) U.S. Cl. CPC A24F 40/60; A24F 40/20; A24F 40/40; A24F 40/50

(57) ABSTRACT

An aerosol-generating device for generating an aerosol from an aerosol-forming substrate is provided, the aerosol-generating device including an OLED display configured to display data indicative of a state of operation of the aerosol-generating device, the OLED display being controlled to display an outer illumination zone partially or wholly surrounding an inner illumination zone, and the OLED display being further controlled to: i) selectively display in one of the outer and the inner illumination zones to generate a predetermined first display conveying first information, and ii) selectively activate the other of the outer and the inner illumination zones to generate a predetermined second display conveying second information.

The present disclosure relates to an aerosol-generating device in which data concerning the progression of an operational phase of the device is visually conveyed to a user of the device.

Aerosol-generating devices configured to generate an aerosol from an aerosol-forming substrate, such as a tobacco containing substrate, are known in the art. Typically, an inhalable aerosol is generated by the transfer of heat from a heat source to a physically separate aerosol-forming substrate or material, which may be located within, around or downstream of the heat source. An aerosol-forming substrate may be a liquid substrate contained in a reservoir. An aerosol-forming substrate may be a solid substrate. An aerosol-forming substrate may be a component part of a separate aerosol-generating article configured to engage with an aerosol-generating device to form an aerosol. During consumption, volatile compounds are released from the aerosol-forming substrate by heat transfer from the heat source and entrained in air drawn through the aerosol-generating article. As the released compounds cool, they condense to form an aerosol that is inhaled by the consumer.

During use of the aerosol-generating device, changes in one or more parameters of the device may occur. It is desired to provide an aerosol-generating device which is able to efficiently convey data concerning the state of the device to a user.

According to an aspect of the present invention, an aerosol-generating device for generating an aerosol from an aerosol-forming substrate comprises an OLED display configured to display data indicative of a state of operation of the aerosol-generating device. The aerosol-generating device may further comprise a controller configured to control the OLED display to display the data regarding operation of the aerosol-generating device.

As aerosol-generating devices become more sophisticated, more data may be available regarding operation of the device, for example data concerning operational states and operational status, data regarding progression through operational states, and data regarding safety of the device. Typically, operational states of an aerosol-generating device are conveyed to a user by use of one or two LEDs, which may illuminate in one or more colour and may flash. Such indication can be, but many users want to be able to configure devices to operate in a manner that suits their usage behaviour and to be able to configure a device to display data accordingly. The use of an OLED display may increase the resolution of data that may be presented to a user, for example data concerning an internal operating state of the device. Use of an OLED display, such as an OLED screen, may also result in a less bulky display compared with a conventional LED display configured to convey a similar resolution of data. This may help maintain a low weight device that can be, for example, carried effortlessly in a user's pocket. In general, use of an OLED display may increase functionality of the aerosol-generating device.

Preferably, the OLED display is or comprises an OLED screen. The OLED screen may be a flexible screen, which may allow the screen to conform to, or form part of, a curved surface of the aerosol-generating device. For example, the OLED display may be a curved OLED screen. The OLED screen is preferably substantially two dimensional, but may be curved or shaped to conform to a surface of the aerosol-generating device, for example the OLED screen may be shaped to conform to a portion of a housing of the aerosol-generating device.

The OLED display may have a concave surface on a first side and a convex surface on a second side, opposite the first side. For example, the concave surface may face inwards towards a longitudinal axis of the device, and the convex surface may face outwards away from the longitudinal axis. Such a display may advantageously conform to a cylindrical surface of an aerosol-generating device.

Preferably, the aerosol-generating device comprises a housing, for example a housing locating internal components of the device such as a controller, a heater, and a power source. At least a portion of the housing may have a curved external surface, for example at least a portion of the housing may be substantially cylindrical. The OLED display may be an OLED screen mounted on, or in, the curved portion of the housing, for example the substantially cylindrical portion of the housing. The device may comprise a housing having a particular external profile and the OLED display may be an OLED screen conforming to a portion of the external profile of the housing. By incorporating an OLED screen that conforms to an external surface of the device, the screen may effectively become part of the housing of the device. This may allow for a screen having a larger area compared to the size of the device than would be allowed if a flat screen needed to be incorporated, for example. It may be able to provide a more ergonomic device by including a conformal OLED screen as part of the external surface of the device.

The aerosol-generating device is preferably a hand-held aerosol-generating device. Preferably, the OLED display is an OLED screen having a surface area defined by width and length dimensions, in which the OLED screen has a maximum width dimension of between 5 mm and 25 mm and a maximum length dimension of between 5 mm and 25 mm. Where the screen is curved, for example to conform to a curved surface, or to form a curved surface, the length and width dimensions may be width and length dimensions that the screen would have in a flat or uncurved condition. The OLED display may comprise an OLED screen having a surface area of between about 20 mm² and about 1600 mm², for example between 30 mm² and 625 mm², for example between 40 mm² and 250 mm², or between 50 mm² and 125 mm².

The OLED display may be an OLED screen having a substantially polygonal shape. For example the OLD screen may have a shape selected from the list of shapes consisting of square, rectangular, circular, oval, rectangular oblong, circular oblong, hexagonal, pentagonal, heptagonal, and octagonal. The OLED display may comprises a display surface or a display portion that has a substantially polygonal shape, for example a shape selected from the list of shapes consisting of square, rectangular, circular, oval, rectangular oblong, circular oblong, hexagonal, pentagonal, heptagonal, and octagonal. A display portion may be a zone or region of the display screen that is configured to display particular data, for example a region of the screen that is configured to display progress of a usage session.

Advantageously, the OLED display may function as a user input. For example the OLED display may be touch sensitive, or comprise at least one touch sensitive zone or portion, to enable user input or interaction. The OLED display may be a user input element having a capacitive touch sensitivity, for example allowing a user to use a finger or a stylus to interact with the device, for example to operate the device, or to navigate user menus, or to select operational options available on the device. By configuring the device such that the OLED display is also a user input, the need for a separate operating button may be eliminated. By eliminating the need for a separate operating button, more area may be available to provide a larger display screen, thereby improving functionality and user experience.

In preferred embodiments, the device may be configured to detect user interaction with the OLED display, for example a user touch on the OLED display, and convert the user interaction into a user interaction signal. The device may be configured to use the user interaction signal to control one or more aspects of the aerosol-generating device. For example, the user interaction signal may be configured to control one or more operational aspects of the aerosol-generating device. The user interaction signal may be configured to control display of information, for example to allow a user to select one or more predetermined modes for display of information, for example one or more indication modes.

The device may be configured to recognise and distinguish 2 or more different user interactions with the OLED display, each of the 2 or more different user interactions being a different user input. For example, the 2 or more user interactions may be selected from the list consisting of a single tap, a double tap, a treble tap, a prolonged touch, a swipe left, a swipe right, a swipe up, a swipe down, and a swipe in a pattern.

The aerosol-generating device preferably comprises a graphic display module configured to drive the OLED display. The graphic display module may be configured to receive instructions from the controller to drive the OLED display. The device may also include a memory configured to store one or more graphic files defining graphics to be displayed by the OLED display. The graphic files may, for example, define one or more logos or emblems to be displayed by the OLED display. The graphic files may define one or more sequences of images to be displayed by the OLED display to indicate a status or progression of an operational event of the aerosol-generating device. The graphic files may define one or more animations to be displayed by the OLED display to indicate a status or progression of an operational event of the aerosol-generating device. The memory may also include instructions for one or more mode of operating the aerosol-generating device. For example, the memory may be programmed with instructions for the controller to allow operation and control of the aerosol-generating device.

Preferably, the OLED display is an OLED screen configured to display in a high resolution, for example in high definition or in ultra-high definition.

The aerosol-generating device may be configured to monitor progress of an event, for example an operational event. The device may comprise a controller configured to control the OLED display to display progress of the operational event. For example, the device may be configured to monitor progress of a usage session during which aerosol is generated, and to display progress of the usage session. The controller may include a memory, or be coupled to a memory, containing instructions for monitoring progress of the usage session. Such a memory may contain instructions for at least one display sequence to display progress of the usage session.

An event may have a duration defined by an event start and an event end. Thus, a usage session may have a duration defined by a usage session start and a usage session end. The aerosol-generating device may be configured to monitor a parameter relating to progress of the event.

The parameter, that is the monitored parameter, may have an initial value at initiation or the start of the event, and a terminal value different to the initial value. A monitored value of the parameter may be used to calculate progress of the parameter between the initial value and the terminal value, and progress of the parameter may then be used to determine progress of the event.

The parameter relating to progress of the event may be a first parameter. The aerosol-generating device may be configured to monitor both the first parameter relating to progress of the event and a second parameter relating to progress of the event, the second parameter being a different parameter to the first parameter. The device may then be configured to determine progress of the event with respect to both the first parameter and the second parameter.

Preferably, the parameter, or one or both of the first parameter and the second parameter, is a user interaction parameter indicative of use of the aerosol-generating device during the event. Preferably, the parameter, or one or both of the first parameter and the second parameter, is a monitored parameter. The parameter, or one or both of the first parameter and the second parameter, may be a cumulative parameter, for example a cumulative value of a monitored parameter over the duration of the event.

Advantageously, progress of the event may be determined by progress of the parameter, or progress of one or both of the first parameter and the second parameter, between its initial value and its terminal value. Progress of the event may be determined as a percentage.

The parameter, the first parameter, or the second parameter may be a parameter selected from the list consisting of time, number of user puffs, cumulative number of user puffs taken during the event, volume of aerosol delivered, cumulative volume of aerosol delivered during the event, energy consumed, cumulative volume of energy consumed during the event, current consumed, cumulative amount of current consumed during the event, temperature, temperature of heating element, temperature of susceptor, resistance of heating element, and user interaction.

Preferably, progress of the event is determined with reference to a first parameter and a second parameter, in which one of the first parameter and the second parameter is time, and the other of the first parameter and the second parameter is selected from the list consisting of number of user puffs, cumulative number of user puffs taken during the event, volume of aerosol delivered, cumulative volume of aerosol delivered during the event, energy consumed, cumulative volume of energy consumed during the event, and current consumed, cumulative amount of current consumed during the event, temperature, temperature of heating element, temperature of susceptor, resistance of heating element, and user interaction.

The OLED display may be controlled to display progress of an event, for example progress of a usage session, as a sequence of display states, for example a sequence of lighting states. Instructions for the sequence of display states, or each sequence of display states may be stored on a memory accessible by the controller. Progress may be displayed as an animated sequence, for example as one or more progressively increasing bars or lines shown on the display, or as one or more progressively decreasing bars or lines shown on the display. The device may be configured to display progress as a sequence of any suitable number of indication states, for example a sequence having between 4 and 144 different indication states. A low number of indication states may provide a relatively crude indication of progress of the event, whereas a large number of indication states may effectively provide a continually changing sequence between 0% progress and 100% progress. The device may be configured to display progress of the event as a sequence of 1st to nth different indication states, n being a number equal to or greater than 4.

The number of indication states is preferably equal to or greater than 7, or equal to or greater than 10. That is, the number n is preferably equal to or greater than 7 or 10. For example, n may be 8, or 9, or 10, or 11, or 12. The number n may be, for example, between 7 and 144. The higher the number, the greater the resolution that the progress of the event or usage session can be presented. However, if the number of states is too high then meaningful differentiation between adjacent states in the sequence may be harder to achieve. Indication in higher numbers of indication states may require increased complexity of the device.

The OLED display may be driven by the controller such that progression through the first to nth indication states involves a corresponding increase in intensity or luminance of light displayed. Alternatively, the OLED display may be driven such that progression through the first to nth indication states involves a corresponding decrease in intensity or luminance of light displayed.

The OLED display may be controlled to display progress of an event as one or more progressively increasing bars or lines tracing the outline of a shape, for example a circle or an oval. For example, using a circular shape, absence of any bar or line forming part of the circle may indicate 0% progression, a semicircle may indicate 50% progression and a full circle may indicate 100% progression. Likewise, the progression sequence may be indicated by an increasing proportion of segments of a circle, progressing from absence of any indication, through an eighth of a circle, a quarter of a circle, three quarters of a circle, to a full circle to indicate 100% progression. Display in such a manner may provide a quick and intuitive way to determine progress.

The sequence of display states may be an increasing sequence, for example increasing amounts of illumination from nothing to a full circle, or a full line or bar. The sequence of display states may also be a decreasing sequence. For example, the OLED display may be controlled to display progress of an event as one or more progressively decreasing bars or lines tracing the outline of a shape, such as a circle, for example in which a full circle indicates 0% progression, a semicircle indicates 50% progression, and absence of a circle indicates 100% progression.

The OLED display may be controlled to display progress of an event in any suitable manner. Advantageously, the device may be programmed such that the progress is displayed as more than one increasing or decreasing element. For example, progress may be displayed as bars or lines tracing the outline of two or more concentric circles. More than one increasing or decreasing element may allow for a more detailed state of progress to be conveyed to a user.

The OLED display may be controlled to display an outer illumination zone partially or wholly surrounding an inner illumination zone; in which the OLED display is controlled to:
i) selectively display in one of the outer and inner illumination zones to generate a predetermined first display conveying first information; and
ii) selectively activate the other of the outer and inner illumination zones to generate a predetermined second display conveying second information.

The outer illumination zone may circumscribe at least 50%, or preferably at least 60%, or preferably at least 70%, or preferably at least 80%, or preferably at least 90%, or preferably all of the perimeter of the inner illumination zone.

The first information may relate to progression of an operational phase of the aerosol-generating device, the second information may relate to a state of the aerosol-generating device, the predetermined first display may be a predetermined sequence or animation, and the predetermined second display may be a predetermined sequence, animation, symbol, logo, or light emission.

The control electronics are preferably configured to generate the predetermined first display and the predetermined second display simultaneously.

In order to facilitate the control of the event, or to facilitate the display of progress of the event, the event, for example the usage session, may divided into n sequential phases, n being a number greater than 4, for example the number greater than 7, for example between 12 and 144 sequential phases, or for example between 18 and 72 sequential phases. Thus, the number of sequential phases, for example the n sequential phases, may be equal to the number of sequential indication states provided by the OLED display, for example the n indication states.

The aerosol-generating device may be configured such that any, or each, of the n sequential phases has a phase duration defined by a phase start and a phase end. The aerosol-generating device may be configured such that any, or each, of the n sequential phases has a maximum phase duration determined by a timer. Any, or each, of the n sequential phases may end when a monitored period of time reaches a predetermined threshold for the phase, if the phase has not ended sooner.

An exemplary aerosol-generating device is configured such that a first phase of n sequential phases has a first phase duration defined by a first phase start and a first phase end, in which the first phase starts at the event start, for example at the usage session start. A second phase of the n sequential phases may have a second phase duration defined by a second phase start and a second phase end, in which the second phase starts at the end of the first phase. The n sequential phases may be defined as a first phase and n−1 subsequent phases, each of the subsequent phases following a preceding phase, in which each of the n−1 subsequent phases has a phase duration defined by a phase start and a phase end, and in which the phase starts at the end of the preceding phase. The event, for example the usage session, preferably ends at the end of the nth phase.

The aerosol-generating device may be configured to monitor a user interaction parameter indicative of use of the aerosol-generating device during the event, for example during the usage session. Advantageously, duration of any, or each, of the n sequential phases may be controlled with reference to the user interaction parameter. A duration of any, or each, of the n sequential phases may be controlled with reference to the user interaction parameter and at least one further parameter.

Preferably the event, for example the usage session, has a maximum duration of between 60 seconds and 600 seconds, for example between 300 seconds and 400 seconds, for example about 360 seconds. Such a maximum duration may replicate the length of a typical smoking session using a conventional cigarette.

Advantageously, the event, for example the usage session, may have a maximum duration of x seconds, x being a number between 100 and 600. For example, the event may be divided into n sequential phases, with a maximum duration of each of the n sequential phases being about x/n seconds.

The event, for example the usage session, may be controlled with respect to a monitored number of user puffs, the usage session having a threshold number of user puffs of between 10 and 14, for example about 12.

In some examples, an aerosol-generating device may be configured to monitor a parameter indicative of aerosol generation during operation of the aerosol-generating device, analyse the monitored parameter to identify a user puff, the user puff defined by a puff start and a puff end, analyse the monitored parameter during the user puff to calculate a puff volume, the puff volume being a volume of aerosol generated during the user puff, and use the puff volume as a parameter relating to progress of the event, for example progress of a usage session. The parameter indicative of aerosol generation may be representative of power supplied by the power supply, for example current, or both current and voltage. Advantageously, the puff volume may be used as a parameter for indicating progress of the usage session. In particular, a usage session may have a threshold value of aerosol that can be delivered and cumulative volume of aerosol generated may be used as a parameter for indicating progress of the usage session.

A measurement of the actual volume of aerosol generated may be complex to implement. Thus, a function of the monitored parameter may be calculated in real time and evaluated to determine a puff volume. Analysis of the monitored parameter may comprise steps of calculating a first characteristic of the monitored parameter and analysing the first characteristic to determine a puff start and a puff stop. Analysis of the monitored parameter may comprise steps of calculating a second characteristic of the monitored parameter and analysing both the first characteristic and the second characteristic to determine the puff start and the puff stop. A puff start may be determined when the first characteristic and the second characteristic satisfy one or more predetermined conditions. Likewise, a puff end may be determined when the first characteristic and the second characteristic satisfy one or more predetermined conditions. Preferably, the first characteristic may be a first moving average value of the monitored parameter computed on a first time window having a first time window duration. The second characteristic may be a second moving average value of the monitored parameter computed on a second time window having a second time window duration, the second time window duration being different to the first time window duration.

In some examples, the event may be a first event, the device further being configured to monitor progress of and display information such as progress in relation to a second event, the second event being different to the first event. For example, the aerosol-generating device may be configured to display progress of a first event and also status of, or progress of, a second event during use of the aerosol-generating device. The aerosol-generating device may comprise an OLED display configured to display progress of the first event as a first sequence of 1st to nth different indication states, and status of, and/or progress of the second event as a second sequence of different indication states n being a number equal to or greater than 7. The device may also comprise a controller configured to monitor progress of the first event and to control the visual indicator to display in an indication state representative of the progress of the first event, and also to monitor the second event and to control the visual indicator to display in an indication state representative of the status and/or progress of the second event. Although the term “a controller” is used, it is intended that this term covers more than one controller if more than one separate controllers are used to control operation of the aerosol-generating device.

Progress of the second event is preferably displayed as a second sequence of 1st to nth different indication states, the second sequence of indication states being different to the first sequence of indication states. By changing the sequence of indication states, the user can easily determine what type of event the device is undergoing and how much progress has been made through the event.

The first event may be an event type selected from the list consisting of a usage session, a heating period, for example, a pre-heating period, a calibration period, a charging period, and a pause period, and the second event may be an event type selected from the list consisting of a usage session, a heating period, for example, a pre-heating period, a calibration period, a charging period, and a pause period, the event type of the second event being different to the event type of the first event.

The controller may be configured to determine initiation of the first event, determine an event type of the first event, monitor progress of the first event, and control the OLED display to display a predetermined sequence of indication states representative of progress of the first event. The controller may also be configured to determine initiation of the second event, determine the event type of the second event, monitor status and/or progress of the second event, and control the OLED display to display a predetermined sequence of indication states representative of status and/or progress of the second event.

The second event may occur after termination of the first event. For example, the first event may be a preheating period and the second event may be a usage session that occurs after completion or termination of the preheating period. For example, the first event may be a calibration period and the second event may be a usage session that occurs after completion or termination of the calibration period.

The second event may occur during a hiatus in the first event. For example, the first event may be a usage session and the second event may be a pause period that occurs during a hiatus in the usage session. For example, the first event may be a usage session and the second event may be a recalibration period that occurs during a hiatus in the usage session.

The device may further be configured to monitor progress of and display progress in relation to a third event, the third event being different to the first event and the second event. In such a case, the third event may be an event type selected from the list consisting of a usage session, a heating period, for example a pre-heating period, a calibration period, a charging period, and a pause period. As an example, the first event may be a preheating event, the second event may be a usage session, and the third event may be a pause event.

An aerosol-generating device may be configured to allow a user to pause an event, for example the first event, during progress of the event and enter a pause period. The device may therefore comprise a memory configured to store progress of the event such that display of progress can be resumed on re-initiation of the event.

Where the device is configured to display information relating to, such as progress of, more than one different type of event, the device may be configured such that different portions of the OLED display are used to present data relating to the different types of event.

An aerosol-generating device may further comprise a user interaction interface, for example an interface selected from the list consisting of a button, a touch sensitive button, a strain sensitive button, a gesture recognition interface, a haptic interface, and an accelerometer. An aerosol-generating device may comprise a power source for supplying energy to generate an aerosol from an aerosol-forming substrate, for example a power source such as a battery.

An aerosol-generating device as described herein may comprise a heater for heating an aerosol-forming substrate, for example a resistance heater or an induction heater. The device may be configured to operate with a solid aerosol-forming substrate. The device may be configured to operate with a liquid aerosol-forming substrate.

Advantageously, the aerosol-generating device may comprise a sensor, for example a sensor for detecting a parameter indicative of progress of the event, for example a sensor for detecting a user interaction parameter.

The aerosol-generating device may comprise a heater for heating an aerosol-forming substrate to form an aerosol. The heater may be an induction heater. An induction heater may comprise an inductor configured to generate a fluctuating magnetic field designed to heat a susceptor. The heater may be a resistance heater.

The heater may comprise a heating element for heating a consumable aerosol-generating article. The heating element may be an internal heater designed to be inserted into a consumable aerosol-generating article, for example a resistive heating element or a susceptor in the form of a pin or blade that can be inserted into an aerosol-forming substrate located within a consumable aerosol-generating article. The heating element may be an external heater designed to heat an external surface of a consumable aerosol-generating article, for example a resistive heating element or a susceptor located at the periphery of, or surrounding, a substrate receiving cavity for receiving the consumable aerosol-generating article.

The aerosol-generating device may comprise a replaceable substrate section containing an aerosol-forming substrate. The replaceable substrate section may form a portion of body of the aerosol-generating device and may itself locate or contain a portion of aerosol-forming substrate for consumption in the device. The replaceable substrate section may be located distal to the proximal end of the device, for example distal to a mouthpiece. The replaceable substrate section may be located proximal to the distal end of the device. The replaceable substrate section may be coupled to one or more other sections forming the body of the aerosol-generating device by coupling means such as screw threads, or bayonet fitting, or magnetic connection, or mechanical latching means such as snap fits or interference fit.

A replaceable substrate section may comprise a reservoir of liquid aerosol-forming substrate. For example, a replaceable substrate section may comprise a reservoir of a liquid comprising nicotine and an aerosol former such as propylene glycol or glycerine. Alternatively, a replaceable substrate section may comprise a container of solid aerosol-forming substrate, or a container of colloidal aerosol-forming substrate such as a gel substrate.

The aerosol-generating device may comprise a replaceable substrate section containing two or more components which form an aerosol when combined.

A replaceable substrate section may comprise an atomizer, such as a heating element for heating the aerosol-forming substrate, or for heating at least one of the two or more components which form an aerosol when combined. Thus, a replaceable substrate section may be a form of cartomizer and include both an aerosol-forming substrate and an atomizing component. The replaceable substrate section would, in such embodiments, preferably include electrical contacts configured to contact corresponding electrical contacts on a battery portion of the aerosol-generating device to provide power for actuation of the atomizer.

In an example, the atomizer may be a resistance heater such as a resistive wire, or a resistive track on a substrate. In other examples, the atomizer may be an inductive susceptor capable of heating when within a fluctuating magnetic field generated by an inductive coil.

The aerosol-generating device may be configured such that power is supplied to the heater to maintain the heater at a predetermined temperature during the usage session.

Power may be supplied to the heater to increase the temperature of a heater element to an operating temperature range for generating an aerosol, the heater element remaining within the operating temperature range until the end of a usage session. Power may be supplied to the heater during a usage session both when a user is taking a puff and when a user is not taking a puff. In such a configuration, the power supplied during a user puff is likely to be greater than that supplied when a user is not talking a puff, as less power with be required to maintain the temperature of the heater between puffs.

An aerosol-generating device may be configured to receive an aerosol-generating article comprising an aerosol-forming substrate. The aerosol-forming substrate may be a solid aerosol-forming substrate. The aerosol-generating device may, for example, comprise a substrate receiving cavity for receiving a consumable aerosol-generating article comprising an aerosol-forming substrate. Examples of aerosol-generating articles include sachets filled with solid aerosol-forming substrates, cigarettes and cigarette-like articles that include an aerosol-forming substrate contained within a wrapper such as a cigarette paper, capsules or containers of liquid aerosol-forming substrate or colloidal aerosol-forming substrate. The consumable aerosol-generating article may comprise a replaceable substrate section containing two or more components which form an aerosol when combined.

A consumable aerosol-generating article may comprise an atomizer, such as a heating element for heating the aerosol-forming substrate, or for heating at least one of the two or more components which form an aerosol when combined. Thus, a consumable aerosol-generating article may be a form of cartomizer and include both an aerosol-forming substrate and an atomizing component. The consumable aerosol-generating article would, in such embodiments, preferably include electrical contacts configured to contact corresponding electrical contacts on a battery portion of the aerosol-generating device to provide power for actuation of the atomizer.

In examples, the atomizer may be a resistance heater such as a resistive wire, or a resistive track on a substrate. In other embodiments, the atomizer may be an inductive susceptor capable of heating when within a fluctuating magnetic field generated by an inductive coil.

A preferred consumable aerosol-generating article may be in the form of a cigarette or cigarette-like article comprising a solid aerosol-forming substrate contained within a wrapper. Preferably such an article includes a mouth end intended to be inserted into a user's mouth for consumption of the article. Preferably, the mouth end includes a filter to emulate a conventional tailored cigarette. Preferably, the consumable aerosol-generating article is configured to interact with an atomizer, preferably a heater, located in the body of the aerosol-generating device. Thus, a heating means such as a resistance heating element may be located in or around the substrate receiving cavity for receiving the consumable aerosol-generating article. The substrate receiving cavity may be located at a proximal end of the device. For example, an opening to the substrate receiving cavity may be located at the proximal end of the device.

Preferably, the aerosol-forming substrate of the aerosol-generating article is a solid aerosol-forming substrate. However, the aerosol-forming substrate may comprise both solid and liquid components. Alternatively, the aerosol-forming substrate may be a liquid aerosol-forming substrate.

Preferably, the aerosol-forming substrate comprises nicotine. More preferably, the aerosol-forming substrate comprises tobacco. Alternatively or in addition, the aerosol-forming substrate may comprise a non-tobacco containing aerosol-forming material.

If the aerosol-forming substrate is a solid aerosol-forming substrate, the solid aerosol-forming substrate may comprise, for example, one or more of: powder, granules, pellets, shreds, strands, strips or sheets containing one or more of: herb leaf, tobacco leaf, tobacco ribs, expanded tobacco and homogenised tobacco.

Optionally, the solid aerosol-forming substrate may contain tobacco or non-tobacco volatile flavour compounds, which are released upon heating of the solid aerosol-forming substrate. The solid aerosol-forming substrate may also contain one or more capsules that, for example, include additional tobacco volatile flavour compounds or non-tobacco volatile flavour compounds and such capsules may melt during heating of the solid aerosol-forming substrate.

Optionally, the solid aerosol-forming substrate may be provided on or embedded in a thermally stable carrier. The carrier may take the form of powder, granules, pellets, shreds, strands, strips or sheets. The solid aerosol-forming substrate may be deposited on the surface of the carrier in the form of, for example, a sheet, foam, gel or slurry. The solid aerosol-forming substrate may be deposited on the entire surface of the carrier, or alternatively, may be deposited in a pattern in order to provide a non-uniform flavour delivery during use.

In a preferred embodiment, the aerosol-forming substrate comprises homogenised tobacco material. As used herein, the term “homogenised tobacco material” refers to a material formed by agglomerating particulate tobacco.

Preferably, the aerosol-forming substrate comprises a gathered sheet of homogenised tobacco material. As used herein, the term “sheet” refers to a laminar element having a width and length substantially greater than the thickness thereof. As used herein, the term “gathered” is used to describe a sheet that is convoluted, folded, or otherwise compressed or constricted substantially transversely to the longitudinal axis of the aerosol-generating article.

Preferably, the aerosol-forming substrate comprises an aerosol former. As used herein, the term “aerosol former” is used to describe any suitable known compound or mixture of compounds that, in use, facilitates formation of an aerosol and that is substantially resistant to thermal degradation at the operating temperature of the aerosol-generating article.

Suitable aerosol-formers are known in the art and include, but are not limited to: polyhydric alcohols, such as propylene glycol, triethylene glycol, 1,3-butanediol and glycerine; esters of polyhydric alcohols, such as glycerol mono-, di- or triacetate; and aliphatic esters of mono-, di- or polycarboxylic acids, such as dimethyl dodecanedioate and dimethyl tetradecanedioate. Preferred aerosol formers are polyhydric alcohols or mixtures thereof, such as propylene glycol, triethylene glycol, 1,3-butanediol and, most preferred, glycerine.

The aerosol-forming substrate may comprise a single aerosol former. Alternatively, the aerosol-forming substrate may comprise a combination of two or more aerosol formers.

An aerosol-generating system may comprise an aerosol-generating as described above and an aerosol-generating article configured to be received by the aerosol-generating device, the aerosol-generating article comprising the aerosol-forming substrate.

An aerosol-generating system may further comprise a charging device for charging the aerosol-generating device. A charging device may comprise a primary power source and may have a docking arrangement configured to engage with the aerosol-generating device.

As used herein, the term “aerosol-generating device” is used to describe a device that interacts with an aerosol-forming substrate of an aerosol-generating article to generate an aerosol. Preferably, the aerosol-generating device is a smoking device that interacts with an aerosol-forming substrate of an aerosol-generating article to generate an aerosol that is directly inhalable into a user's lungs thorough the user's mouth. The aerosol-generating device may be a holder for an aerosol-generating article. Preferably, the aerosol-generating article is a smoking article that generates an aerosol that is directly inhalable into a user's lungs through the user's mouth. More preferably, the aerosol-generating article is an article that generates a nicotine-containing aerosol that is directly inhalable into a user's lungs through the user's mouth. The aerosol-generating article may be an article that generates a nicotine-free aerosol that is directly inhalable into a user's lungs through the user's mouth.

As used herein, the term ‘aerosol-generating article’ refers to an article comprising an aerosol-forming substrate capable of releasing volatile compounds, which can form an aerosol. In certain embodiments, the aerosol-generating article may comprise an aerosol-forming substrate capable of releasing upon heating volatile compounds, which can form an aerosol.

As used herein, the term “aerosol-forming substrate” denotes a substrate consisting of or comprising an aerosol-forming material that is capable of releasing volatile compounds upon heating to generate an aerosol. The aerosol may comprise nicotine. The aerosol may be a nicotine-free aerosol comprising one or more inhalable substances, but not comprising nicotine.

As used herein, the term ‘usage session’ refers to an operational period of the aerosol-generating device having a finite duration. A usage session may be initiated by the action of a user. A usage session may be terminated after a predetermined period of time has elapsed from the initiation of the usage session. A usage session may be terminated after a monitored parameter has reached a threshold during the usage session. Typically, a usage session has a duration that allows a user to enjoy a single user experience. For example, in certain aerosol-generating devices, a usage session may have a duration that allows the user to consume a single disposable aerosol-generating article. After a usage session has been terminated, further action is required by a user to initiate a subsequent usage session.

As used herein, the term ‘OLED display’ refers to an element using one or more organic light-emitting diode (OLED), for example an OLED screen, to emit a visual indication.

The invention is defined in the claims. However, below there is provided a non-exhaustive list of non-limiting examples. Any one or more of the features of these examples may be combined with any one or more features of another example, embodiment, or aspect described herein.

Ex1. An aerosol-generating device for generating an aerosol from an aerosol-forming substrate, the aerosol-generating device comprising an OLED display configured to display data indicative of a state of operation of the aerosol-generating device.

Ex2. An aerosol-generating device according to example Ex1 further comprising a controller configured to control the OLED display to display the data regarding operation of the aerosol-generating device.

Ex3. An aerosol-generating device according to any preceding example in which the OLED display is an OLED screen.

Ex4. An aerosol-generating device according to any preceding example in which the OLED display is a flexible OLED screen and/or a curved OLED screen.

Ex4a. An aerosol-generating device according to any preceding example wherein the OLED display has a concave surface on a first side and a convex surface on a second side, opposite the first side.

Ex4b. An aerosol-generating device according to example Ex4a wherein the concave surface faces inwards towards a longitudinal axis of the device, and the convex surface faces outwards away from the longitudinal axis.

Ex5. An aerosol-generating device according to any preceding device in which the device comprises a housing, at least a portion of the housing having a curved surface, for example at least a portion of the housing being substantially cylindrical, in which the OLED display is an OLED screen mounted on, or in, the portion of the housing.

Ex6. An aerosol-generating device according to any preceding example in which the device comprises a housing having an external profile and the OLED display is an OLED screen conforming to a portion of the external profile of the housing.

Ex7. An aerosol-generating device according to any preceding example in which the OLED display is an OLED screen having a surface area defined by width and length dimensions, in which the OLED screen has a maximum width dimension of between 5 mm and 25 mm and a maximum length dimension of between 5 mm and 25 mm.

EX8. An aerosol-generating device according to any preceding example in which the OLED display is an OLED screen having a surface area of between about 20 mm² and about 625 mm².

Ex9. An aerosol-generating device according to any preceding example in which the OLED display is an OLED screen having a substantially polygonal shape, for example a shape selected from the list of shapes consisting of square, rectangular, circular, oval, rectangular oblong, circular oblong, hexagonal, pentagonal, heptagonal, and octagonal.

Ex9a. An aerosol-generating device according to any preceding example in which the OLED display comprises a display surface substantially polygonal shape, for example a shape selected from the list of shapes consisting of square, rectangular, circular, oval, rectangular oblong, circular oblong, hexagonal, pentagonal, heptagonal, and octagonal.

Ex10. An aerosol-generating device according to any preceding example in which the OLED display is a user input, for example in which the OLED display is touch sensitive to enable user input.

Ex11. An aerosol-generating device according to example Ex10 in which the OLED is a user input element having a capacitive touch sensitivity.

Ex12. An aerosol-generating device according to any preceding example in which the device is configured to detect user interaction with the OLED display, for example a user touch on the OLED display, and convert the user interaction into a user interaction signal, the device further being configured to use the user interaction signal to control one or more aspects of the aerosol-generating device.

Ex13. An aerosol-generating device according to example Ex12 in which the user interaction signal is configured to control one or more operational aspects of the aerosol-generating device.

Ex14. An aerosol-generating device according to example EX12 or Ex13 in which the user interaction signal is configured to control display of information, for example to allow a user to select one or more predetermined modes for display of information.

Ex15. An aerosol-generating device according to any of examples Ex10 to Ex14 in which the device is configured to recognise and distinguish 2 or more different user interactions with the OLED display, each of the 2 or more different user interactions being a different user input.

Ex16. An aerosol-generating device according to example Ex15 in which the 2 or more user interactions are selected from the list consisting of a single tap, a double tap, a treble tap, a prolonged touch, a swipe left, a swipe right, a swipe up, a swipe down, and a swipe in a pattern.

Ex17. An aerosol-generating device according to any preceding example in which the device comprises a graphic display module configured to drive the OLED display.

Ex18. An aerosol-generating device according to any preceding example in which the device includes a memory configured to store one or more graphic files defining graphics to be displayed by the OLED display.

Ex19. An aerosol-generating device according to example Ex18 in which the graphic files define one or more logos or emblems to be displayed by the OLED display.

Ex20. An aerosol-generating device according to example Ex18 or Ex19 in which the graphic files define one or more sequences of images to be displayed by the OLED display to indicate a status or progression of an operational event of the aerosol-generating device.

Ex21. An aerosol-generating device according to any of examples Ex18 to Ex20 in which the graphic files define one or more animations to be displayed by the OLED display to indicate a status or progression of an operational event of the aerosol-generating device.

Ex22. An aerosol-generating device according to any preceding example in which the OLED display is an OLED screen configured to display in a high resolution, for example in high definition or in ultra-high definition.

Ex23. An aerosol-generating device according to any preceding example in which the device is configured to monitor progress of an operational event, the device comprising a controller configured to control the OLED display to display progress of the operational event.

Ex24. An aerosol-generating device according to example Ex23 in which the device is configured to monitor progress of a usage session during which aerosol is generated and to display progress of the usage session.

Ex25. An aerosol-generating device according to example Ex24 in which the usage session has a duration defined by a usage session start and a usage session end.

Ex26. An aerosol-generating device according to any of examples Ex23 to Ex25 in which the aerosol-generating device is configured to monitor a parameter relating to progress of the event.

Ex27. An aerosol-generating device according to example Ex26, the parameter having an initial value at initiation or the start of the event, and a terminal value different to the initial value.

Ex28. An aerosol-generating device according to example Ex27, a monitored value of the parameter being used to calculate progress of the parameter between the initial value and the terminal value, progress of the parameter being used to determine progress of the event.

Ex29. An aerosol-generating device according to any of examples Ex26 to Ex28 in which, the parameter relating to progress of the event is a first parameter.

Ex30. An aerosol-generating device according to example Ex29 in which, the aerosol-generating device is configured to monitor both the first parameter relating to progress of the event and a second parameter relating to progress of the event, the second parameter being a different parameter to the first parameter.

Ex31. An aerosol-generating device according to example Ex30 in which the device is configured to determine progress of the event with respect to both the first parameter and the second parameter.

Ex32. An aerosol-generating device according to any of examples Ex23 to Ex31 in which the event has an event start and an event end, a duration of the event defined by the event start and the event end.

Ex33. An aerosol-generating device according to any of examples Ex26 to Ex32 in which the parameter, or one or both of the first parameter and the second parameter, is a user interaction parameter indicative of use of the aerosol-generating device during the event.

Ex34. An aerosol-generating device according to any of examples Ex26 to Ex33 in which the parameter, or one or both of the first parameter and the second parameter, is a monitored parameter.

Ex35. An aerosol-generating device according to any of examples Ex26 to Ex34 in which the parameter, or one or both of the first parameter and the second parameter, is a cumulative parameter, for example a cumulative value of a monitored parameter over the duration of the event.

Ex36. An aerosol-generating device according to any of examples Ex26 to Ex35 in which the progress of the event is determined by progress of the parameter, or progress of one or both of the first parameter and the second parameter, between its initial value and its terminal value.

Ex37. An aerosol-generating device according to example Ex36 in which progress of the event is determined as a percentage.

Ex38. An aerosol-generating device according to any of examples Ex26 to Ex37 in which the parameter, the first parameter, or the second parameter is time.

Ex39. An aerosol-generating device according to any of examples Ex26 to Ex38 in which the parameter, the first parameter, or the second parameter is selected from the list consisting of time, number of user puffs, cumulative number of user puffs taken during the event, volume of aerosol delivered, cumulative volume of aerosol delivered during the event, energy consumed cumulative volume of energy consumed during the event, and current consumed, cumulative amount of current consumed during the event, temperature, temperature of heating element, temperature of susceptor, resistance of heating element, and user interaction.

Ex40. An aerosol-generating device according to any of examples Ex26 to Ex39 in which progress of the event is determined with reference to a first parameter and a second parameter, in which one of the first parameter and the second parameter is time, and the other of the first parameter and the second parameter is selected from the list consisting of number of user puffs, cumulative number of user puffs taken during the event, volume of aerosol delivered, cumulative volume of aerosol delivered during the event, energy consumed cumulative volume of energy consumed during the event, and current consumed, cumulative amount of current consumed during the event, temperature, temperature of heating element, temperature of susceptor, resistance of heating element, and user interaction.

Ex41. An aerosol-generating device according to any preceding example in which the OLED display is controlled to display progress of an event, for example a usage session, as a sequence of lighting states.

Ex42. An aerosol-generating device according to any preceding example in which the OLED display is controlled to display progress of an event, for example a usage session, as an animated sequence.

Ex43. An aerosol-generating device according to any of examples Ex24 to Ex42 in which the OLED display is controlled to display progress of an event as one or more progressively increasing bars or lines.

Ex44. An aerosol-generating device according to any of examples Ex24 to Ex43 in which the OLED display is controlled to display progress of an event as one or more progressively decreasing bars or lines.

Ex45. An aerosol-generating device according to any of examples Ex24 to Ex44 in which the OLED display is controlled to display progress of an event as one or more progressively increasing bars or lines tracing the outline of a circle, for example in which absence of a circle indicates 0% progression, a semicircle indicates 50% progression and a full circle indicates 100% progression.

Ex46. An aerosol-generating device according to any of examples Ex24 to Ex45 in which the OLED display is controlled to display progress of an event as one or more progressively decreasing bars or lines tracing the outline of a circle, for example in which absence of a circle indicates 100% progression, a semicircle indicates 50% progression and a full circle indicates 0% progression.

Ex47. An aerosol-generating device according to any of example Ex24 to Ex46 in which the OLED display is controlled to display progress of an event as bars or lines tracing the outline of concentric circles.

Ex48. An aerosol-generating device according to any preceding example in which the OLED display is controlled to display an outer illumination zone partially or wholly surrounding an inner illumination zone;
in which the OLED display is controlled to:
i) selectively display in one of the outer and inner illumination zones to generate a predetermined first display conveying first information; and
ii) selectively activate the other of the outer and inner illumination zones to generate a predetermined second display conveying second information.

Ex49. An aerosol-generating device according to example Ex48, in which the outer illumination zone circumscribes at least 50%, or preferably at least 60%, or preferably at least 70%, or preferably at least 80%, or preferably at least 90%, or preferably all of the perimeter of the inner illumination zone.

Ex50. An aerosol-generating device according to example Ex48 or Ex49, in which the first information relates to progression of an operational phase of the aerosol-generating device, the second information relates to a state of the aerosol-generating device, the predetermined first display is a predetermined sequence or animation, and the predetermined second display is a predetermined symbol, logo, or light emission.

Ex51. An aerosol-generating device according to example Ex50, in which the control electronics are configured to generate the predetermined first display and the predetermined second display simultaneously.

Examples will now be further described with reference to the figures, in which:

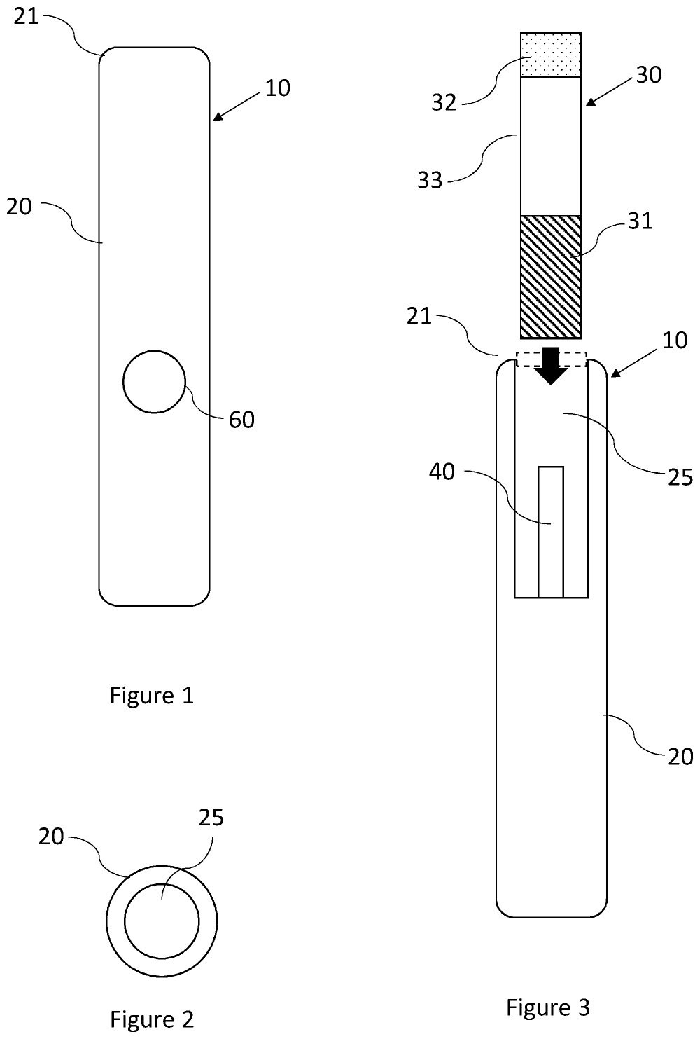

FIG. 1 illustrates a schematic side view of an aerosol-generating device;

FIG. 2 illustrates a schematic upper end view of the aerosol-generating device of FIG. 1;

FIG. 3 illustrates a schematic cross-sectional side view of the aerosol-generating device of FIG. 1 and an aerosol-generating article for use with the device;

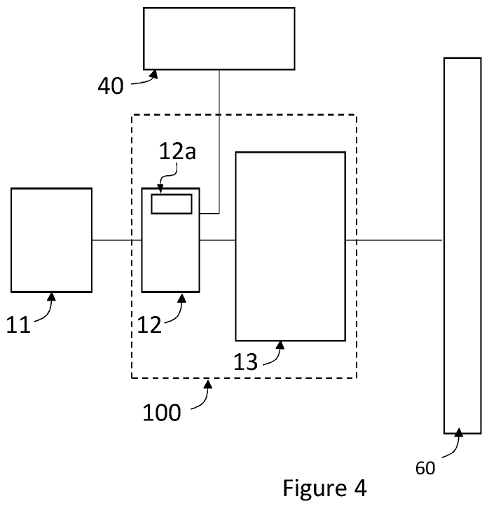

FIG. 4 is a block diagram providing a schematic illustration of various electronic components of the aerosol-generating device of FIGS. 1 to 3 and their interactions;

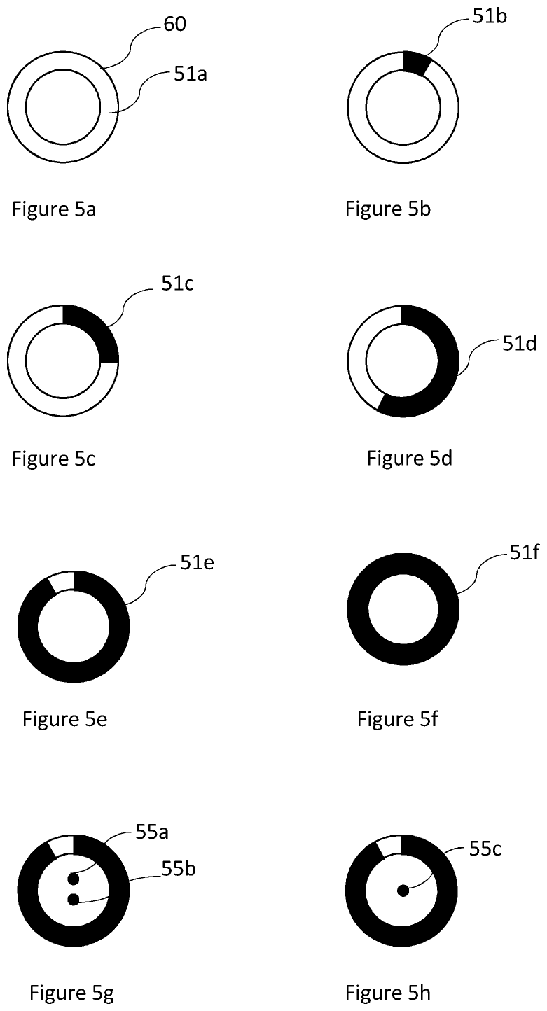

FIGS. 5a to 5h provide schematic figures illustrating the operation of an OLED screen provided on the aerosol-generating device of FIGS. 1 to 4, in particular with progression through a usage session;

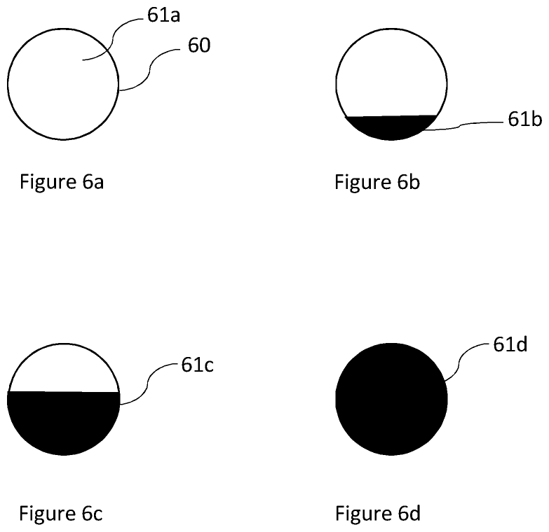

FIGS. 6a to 6d provide schematic figures illustrating a further example of the operation of an OLED screen provided on the aerosol-generating device of FIGS. 1 to 4, with progression through a usage session;

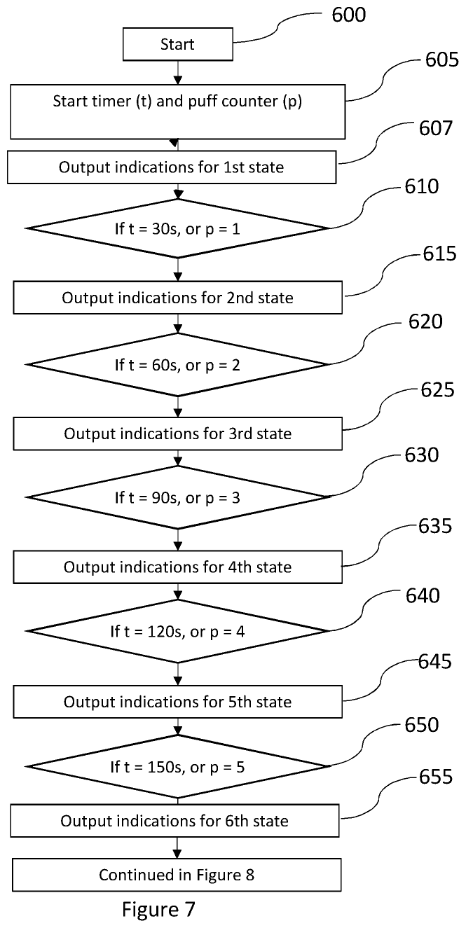

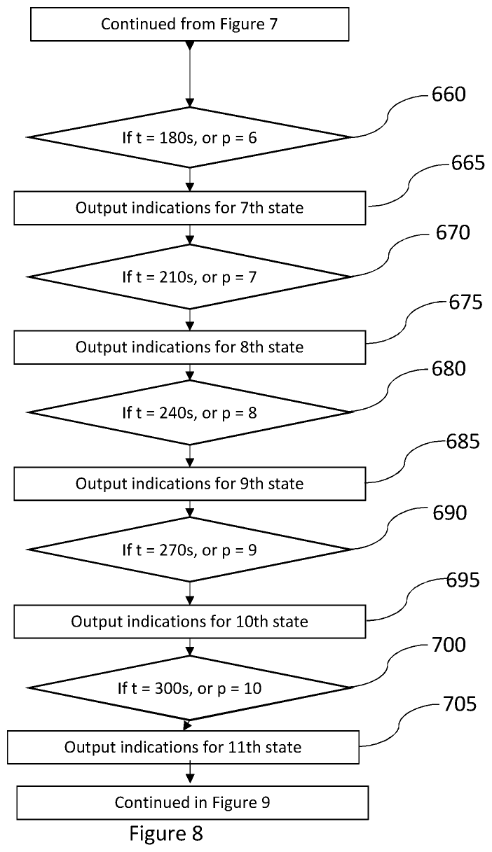

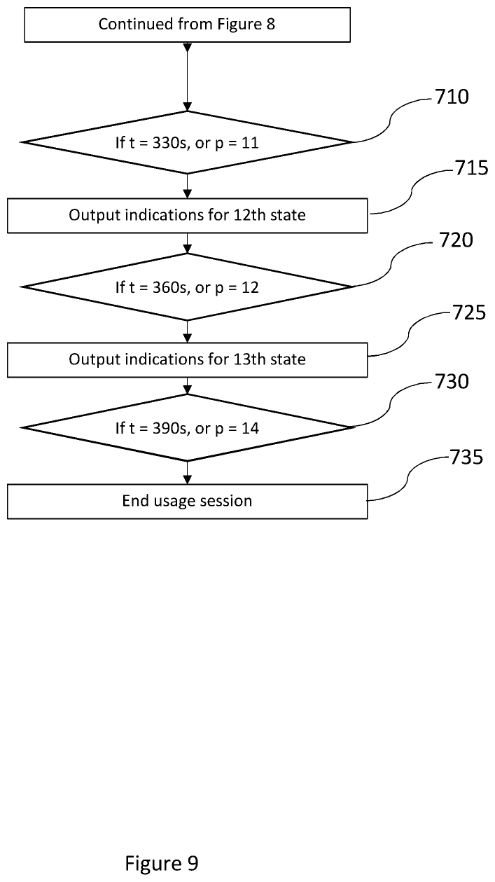

FIGS. 7, 8, and 9 illustrate a flow diagram illustrating method steps involved in determining and indicating progress of a usage session to a user, where progress is determined by time and puff count.

As illustrated in FIGS. 1 to 3, an exemplary aerosol-generating device 10 is a hand-held aerosol generating device, and has an elongate shape defined by a housing 20 that is substantially circularly cylindrical in form. The aerosol-generating device 10 comprises an open cavity 25 located at a proximal end 21 of the housing 20 for receiving an aerosol-generating article 30 comprising an aerosol-forming substrate 31. The aerosol-generating device 10 further comprises a battery (not shown) located within the housing 20 of the device, and an electrically operated heater 40 arranged to heat at least an aerosol-forming substrate portion 31 of an aerosol-generating article 30 when the aerosol-generating article 30 is received in the cavity 25.

The aerosol-generating device is configured to receive a consumable aerosol-generating article 30. The aerosol-generating article 30 is in the form of a cylindrical rod and comprises an aerosol-forming substrate 31. The aerosol-forming substrate is a solid aerosol-forming substrate comprising tobacco. The aerosol-generating article 30 further comprises a mouthpiece such as a filter 32 arranged in coaxial alignment with the aerosol-forming substrate within the cylindrical rod. The aerosol-generating article 30 has a diameter substantially equal to the diameter of the cavity 25 of the device 10 and a length longer than a depth of the cavity 25, such that when the article 30 is received in the cavity 25 of the device 10, the mouthpiece 32 extends out of the cavity 25 and may be drawn on by a user, similarly to a conventional cigarette. In preferred embodiments, the aerosol-generating article is 45 mm long and 7.2 mm in diameter.

In use, a user inserts the article 30 into the cavity 25 of the aerosol-generating device 10 and turns on the device 10 by tapping a touch sensitive OLED screen 60 to activate the heater 40 to start a usage session. The touch sensitive OLED screen 60 functions as both a display and a user interface. The heater 40 heats the aerosol-forming substrate of the article 30 such that volatile compounds of the aerosol-forming substrate 31 are released and atomised to form an aerosol. The user draws on the mouthpiece of the article 30 and inhales the aerosol generated from the heated aerosol-forming substrate. After activation, the temperature of the heater 40 increases from an ambient temperature to a predetermined temperature for heating the aerosol-forming substrate. Control electronics of the device 10 supply power to the heater from the battery to maintain the temperature of the heater at an approximately constant level as a user puffs on the aerosol-generating article 30. The heater continues to heat the aerosol-generating article until an end of the usage session, when the heater is deactivated and cools.

At the end of the usage session, the article 30 is removed from the device 10 for disposal, and the device 10 may be coupled to an external power source for charging of the battery of the device 10.

The aerosol-generating device comprises a light emitting indicator or display in the form of the touch sensitive OLED screen 60. The OLED screen 60 is incorporated into the housing 20 of the aerosol-generating device 10.

FIG. 4 provides a schematic illustration of various electronic components of the aerosol-generating device and their interactions.

A microcontroller or controller 12, located within the housing 20, is connected to a battery 11, a heater 40, a timer 430, an OLED driver 13 and the OLED screen 60. The battery 11 supplies energy to heat the heater 40 and to operate other electrical components. The battery 11 has, when fully charged, sufficient energy to power two complete usage session of the aerosol-generating device. The battery 11 is a rechargeable battery and can be connected to an external power supply to be recharged.

The heater 40 converts energy supplied by the battery into heat to heat the aerosol-generating device sufficiently to form an aerosol. During operation, the controller 12 controls supply of energy from the battery to maintain the heater at a substantially constant aerosol-generating temperature.

The timer 430 provides timing signals to the controller 12. The OLED screen 60 is configured to receive user inputs and send signals to the controller, and to emit visual indications in response to control signals received from the controller 12. The battery 11 and the controller 12 are coupled to each other and located within the housing 20. The controller 12 also incorporates a memory module 12a. The controller 12 is in turn coupled to both the heater element 40 and the OLED driver 13. The controller 12 and OLED driver 13 collectively form a control electronics section 100 of the aerosol-generating device 10. The memory module 12a contains instructions for execution by the controller 12 during use of the device 10. The instructions stored in the memory module 12a include criteria determining the duration of a usage session, plus other data and information relevant to control and operation of the aerosol-generating device 10. When activated, the controller 12 accesses the instructions contained in the memory module 12a and controls a supply of energy from the battery 11 to the heater element 40 according to the instructions contained in the memory module 12a. The controller 12 also controls a supply of energy to the OLED driver 13 and the OLED screen 60.

The aerosol-generating device 10 of this specific embodiment is configured to determine and monitor progress of a usage session, and to output visual indication of the progress of the usage session as a continuous sequence of different indication states. The sequence of different indication states may flow together as an animation to indicate progress.

A simple exemplary sequence of 13 different indication states is illustrated with reference to FIGS. 5a to 5f. The OLED display screen 60 has a circular shape with dimensions of 9 mm by 9 mm (i.e. diameter of 9 mm). The OLED screen is configured to illustrate progress of an event such as a usage session as a gradually growing ring of light, for example a waxing ring of light. In the context used herein, the term “waxing” means gradually or progressively increasing. In an initial stage, when an event has just started and no progress has been made, the ring is unilluminated 51a. In a final stage, when the event has completed, the ring is fully illuminated 51f.

As shown in FIG. 5a, the OLED screen 60 illustrates a first state of a progression, in which an indication ring is unilluminated 51a. While the unilluminated ring is represented in FIG. 5a for illustrative purposes, it is noted that there may be no indication of the ring if it is not illuminated.

In a second indication state, as shown in FIG. 5b, an arcuate section of a ring of about 30 degrees 51b is illuminated on the OLED screen 60.

It is noted that the OLED screen can be configured to display in many different manners. For example, in some embodiments the indication ring illustrated in FIG. 5a may be illuminated as a first colour 51a when the device is in the first indication state, and that colour may gradually change to a second colour 51b as the event undergoes its progression through different indication states.

In a third indication state, an arcuate section of a ring of about 60 degrees is illuminated on the OLED screen.

In a fourth indication state, as shown in FIG. 5c, an arcuate section of a ring of about 90 degrees 51c is illuminated on the OLED screen 60.

In a fifth indication state, an arcuate section of a ring of about 120 degrees is illuminated on the OLED screen.

In a sixth indication state, an arcuate section of a ring of about 150 degrees is illuminated on the OLED screen.

In a seventh indication state, an arcuate section of a ring of about 180 degrees is illuminated on the OLED screen.

In an eighth indication state, as shown in FIG. 5d, an arcuate section of a ring of about 210 degrees 51d is illuminated on the OLED screen 60.

In a ninth indication state, an arcuate section of a ring of about 240 degrees is illuminated on the OLED screen.

In a tenth indication state, an arcuate section of a ring of about 270 degrees is illuminated on the OLED screen.

In an eleventh indication state, an arcuate section of a ring of about 300 degrees is illuminated on the OLED screen.

In a twelfth indication state, as shown in FIG. 5e, an arcuate section of a ring of about 330 degrees 51e is illuminated on the OLED screen 60.

In a thirteenth indication state, as shown in FIG. 5f, an arcuate section of a ring of about 360 degrees 51f is illuminated on the OLED screen 60, that is a complete ring is illuminated to indicate 100% progression.

This same information can be represented in table form, as seen in table 1 below.

TABLE 1
Representation of a 13 state progression implemented as a
waxing ring on an OLED screen

| | Degrees of illumination of |
|---|---|
| | indication ring on OLED |
| Indication State | screen |
| First | 0 |
| Second | 30 |
| Third | 60 |
| Fourth | 90 |
| Fifth | 120 |
| Sixth | 150 |
| Seventh | 180 |
| Eighth | 210 |
| Ninth | 240 |
| Tenth | 270 |
| Eleventh | 300 |
| Twelfth | 330 |
| Thirteenth | 360 |

The example described above has 13 indication states including the state in which there is no illumination. It is clear that the resolution of indication may be improved by increasing the number of states. There may be, for example, no perceptible jump between different indication states, the indication ring illustrated in FIG. 5a to FIG. 5f appearing to grow continually as the event progresses.

While a gradually increasing or waxing ring may provide indication of progress, it should be clear that a gradually decreasing or waning ring may achieve the same purpose.

Use of a ring as an indication of progress may provide some advantage, as space within the ring may be used for other indications. For example, the OLED screen 60 may be configured to display data relating to use of the aerosol-generating device, for example number of usage sessions remaining. As illustrated in FIG. 5g, indication marks 55a 55b may be positioned within a progression ring to indicate 2 remaining usage sessions. As illustrated in FIG. 5h, a single indication mark 55c may be positioned within a progression ring to indicate 1 remaining usage session.

In a further simple sequence to indicate progression, a circular indication zone 61a of the OLED screen 60 may progressively fill with illumination to indicate progression of an event such as a usage session or a preheating operation. As before, this progression may be broken down into any number of indication stages, for example 11 stages.

In FIG. 6a, the OLED screen 60 is illustrated in a first or unilluminated stage indicating 0% progression. As progression of the event gets underway, the circular indication zone fills with increasing illumination, the percentage illumination being an indication of progress of the event. For example, FIG. 6b illustrates an OLED screen in which a lower 20% of the screen is illuminated 61b, indicating a progression of 20% through the event. FIG. 6c illustrates an OLED screen in which a lower 60% of the screen is illuminated 61c, indicating a progression of 60% through the event. FIG. 6d illustrates an OLED screen in which the entire screen is illuminated 61d, indicating a progression of 100% through the event.

Such a progression in 11 indication states, including an unilluminated state, is represented in table 2. An increase in the number of indication states would result in a decrease in the increase in illumination between successive states, and a resulting increase in the resolution of display that is possible.

TABLE 2
Representation of an 11 state progression implemented
as a fill of illumination on an OLED screen.

| | Percentage illumination of |
|---|---|
| Indication State | an OLED screen |
| First | 0 |
| Second | 10 |
| Third | 20 |
| Fourth | 30 |
| Fifth | 40 |
| Sixth | 50 |
| Seventh | 60 |
| Eighth | 70 |
| Ninth | 80 |
| Tenth | 90 |
| Eleventh | 100 |

When fully charged, the battery can provide sufficient energy for at least one full usage sessions. The battery may provide sufficient energy for two or more usage session (for instance, twenty usage sessions).

An aerosol-generating article for use with the device has a finite quantity of aerosol-forming substrate and, thus, a usage session needs to have a finite duration to prevent a user trying to produce aerosol when the aerosol-forming substrate has been depleted. A usage session is configured to have a maximum duration determined by a period of time from the start of the usage session. A usage session is also configured to have a duration of less than the maximum duration if a user interaction parameter recorded during the usage session reaches a threshold before the maximum duration as determined by the timer.

In a specific embodiment the user interaction parameter is number of puffs taken by the user during the usage session. Thus, in a specific embodiment, the aerosol-generating device is configured such that each usage session has a duration of 6.5 minutes (390 seconds) from initiation of the usage session, or 14 puffs taken by the user if 14 puffs are taken within 6.5 minutes from initiation of the usage session. The exact time or number of puffs may be varied to any suitable value. For example the session may have a duration limited to 6 minutes, or to 5.5 minutes. As a further example, the number of allowed puffs may be limited to 13 or 12.

During a usage session, a user may wish to have an indication of progress through the usage session. For example, the user may wish to know approximately how many puffs he has remaining, or approximately how much time there remains in the usage session.

The controller comprises a puff counter to monitor number of puffs taken during a usage session. Number of puffs taken by the user is determined by monitoring power supplied to the heater during the usage session. When a user takes a puff, the flow of air cools the heater and, therefore, a greater amount of energy is supplied by the battery to maintain the temperature of the heater at its operational temperature. Thus, by monitoring power supplied by the heater, the controller is able to determine the number of puffs taken during a usage session.

In order to monitor progress, a usage session is split into a number of sequential phases starting with a first phase starting when the usage session starts and ending with a final phase when the usage session ends, passage from one phase to a next phase being determined by time and puff number in the same way as the usage session. Each phase is deemed to have ended when criteria for that phase meet a predetermined threshold. As the usage session progresses through its sequential phases, the controller instructs the light emitting indicator to emit signals indicative of each successive phase. Thus, a user knows approximately the progress of the usage session.

In a specific example, a usage session may be broken into thirteen sequential phases for indication purposes. FIGS. 7, 8, and 9 show a flow diagram illustrating the method steps involved in indicating progress of a usage session to a user.

Step 600: The user inserts an aerosol-generating article 30 into the cavity 25 of the device 10 and initiates a usage session by tapping the touch sensitive OLED display 60.

Step 605: The timer is initiated to record time elapsed during the usage session and the puff counter is initiated to record number of puffs taken during the usage session.

Step 607: A first phase of the usage session is deemed to have started when the usage session started. The controller instructs the OLED display 60 to output indications of a first, or initial, state of progress of the usage session.

Step 610: The first phase ends and the second phase begins after 30 seconds have elapsed from the start of the usage session, or after a user has taken 1 puff since the start of the usage session, if that puff is taken before 30 seconds has elapsed from the start of the usage session.

Step 615: The controller instructs the OLED display 60 to output indications of a second state of progress of the usage session.

Step 620: The second phase ends and the third phase begins after 60 seconds have elapsed from the start of the usage session, or after a user has taken 2 puffs since the start of the usage session, if those puffs are taken before 60 seconds has elapsed from the start of the usage session.

Step 625: The controller instructs the OLED display 60 to output indications of a third state of progress of the usage session.

Step 630: The third phase ends and the fourth phase begins after 90 seconds have elapsed from the start of the usage session, or after a user has taken 3 puffs since the start of the usage session, if those puffs are taken before 90 seconds has elapsed from the start of the usage session.

Step 635: The controller instructs the OLED display 60 to output indications of a fourth state of progress of the usage session.

Step 640: The fourth phase ends and the fifth phase begins after 120 seconds have elapsed from the start of the usage session, or after a user has taken 4 puffs since the start of the usage session, if those puffs are taken before 120 seconds has elapsed from the start of the usage session.

Step 645: The controller instructs the OLED display 60 to output indications of a fifth state of progress of the usage session.

Step 650: The fifth phase ends and the sixth phase begins after 150 seconds have elapsed from the start of the usage session, or after a user has taken 5 puffs since the start of the usage session, if those puffs are taken before 150 seconds has elapsed from the start of the usage session.

Step 655: The controller instructs the OLED display 60 to output indications of a sixth state of progress of the usage session.

Step 660: The sixth phase ends and the seventh phase begins after 180 seconds have elapsed from the start of the usage session, or after a user has taken 6 puffs since the start of the usage session, if those puffs are taken before 180 seconds has elapsed from the start of the usage session.

Step 665: The controller instructs the OLED display 60 to output indications of a seventh state of progress of the usage session.

Step 670: The seventh phase ends and the eighth phase begins after 210 seconds have elapsed from the start of the usage session, or after a user has taken 7 puffs since the start of the usage session, if those puffs are taken before 210 seconds has elapsed from the start of the usage session.

Step 675: The controller instructs the OLED display 60 to output indications of an eighth state of progress of the usage session.

Step 680: The eighth phase ends and the ninth phase begins after 240 seconds have elapsed from the start of the usage session, or after a user has taken 8 puffs since the start of the usage session, if those puffs are taken before 240 seconds has elapsed from the start of the usage session.

Step 685: The controller instructs the OLED display 60 to output indications of an ninth state of progress of the usage session.

Step 690: The ninth phase ends and the tenth phase begins after 270 seconds have elapsed from the start of the usage session, or after a user has taken 9 puffs since the start of the usage session, if those puffs are taken before 270 seconds has elapsed from the start of the usage session.

Step 695: The controller instructs the OLED display 60 to output indications of an tenth state of progress of the usage session.

Step 700: The tenth phase ends and the eleventh phase begins after 300 seconds have elapsed from the start of the usage session, or after a user has taken 10 puffs since the start of the usage session, if those puffs are taken before 300 seconds has elapsed from the start of the usage session.

Step 705: The controller instructs the OLED display 60 to output indications of an eleventh state of progress of the usage session.

Step 710: The eleventh phase ends and the twelfth phase begins after 330 seconds have elapsed from the start of the usage session, or after a user has taken 11 puffs since the start of the usage session, if those puffs are taken before 330 seconds has elapsed from the start of the usage session.

Step 715: The controller instructs the OLED display 60 to output indications of an twelfth state of progress of the usage session.

Step 720: The twelfth phase ends and the thirteenth, or final, phase begins after 360 seconds have elapsed from the start of the usage session, or after a user has taken 11 puffs since the start of the usage session, if those puffs are taken before 360 seconds has elapsed from the start of the usage session.

Step 725: The controller instructs the OLED display 60 to output indications of a thirteen state of progress of the usage session.

Step 730: The thirteen phase is the final phase of the usage session. During the final phase the user may take two puffs, taking the total number of puffs during the usage session to 14. The indication of the thirteenth state of progress may include further indication that the thirteenth phase is the final phase. For example, the output indication may include a change of colour as well as the change in overall intensity representing progress. The thirteenth phase ends after 390 seconds have elapsed from the start of the usage session, or after a user has taken 14 puffs since the start of the usage session, if those puffs are taken before 390 seconds has elapsed from the start of the usage session.

Step 735: The usage session ends.

The same information as set out in FIGS. 7 to 9 may be presented in table form, as for example in table 3 below.

TABLE 3
Phases of a usage session including displayed indication state and
criteria for ending each phase in terms of puff number or time from
the start of the usage session.

| | | Criteria for ending phase | | |
|---|---|---|---|---|
| Phase | Displayed | Puff | | Time |
| No. | Indication State | No. | | (s) |
| 1 | 1 | 1 | or | 30 |
| 2 | 2 | 2 | or | 60 |
| 3 | 3 | 3 | or | 90 |
| 4 | 4 | 4 | or | 120 |
| 5 | 5 | 5 | or | 150 |
| 6 | 6 | 6 | or | 180 |
| 7 | 7 | 7 | or | 210 |
| 8 | 8 | 8 | or | 240 |
| 9 | 9 | 9 | or | 270 |
| 10 | 10 | 10 | or | 300 |
| 11 | 11 | 11 | or | 330 |
| 12 | 12 | 12 | or | 360 |
| 13 | 13 | 14 | or | 390 |

The example above divides a usage session into thirteen sequential phases, each phase ending when certain criteria regarding number of puffs taken or time elapsed are fulfilled. Each of the thirteen sequential phases can be represented by one of thirteen sequential indication states. As an example, where the aerosol-generating device has an indicator in the form of an OLED screen, the thirteen sequential indication states may be those set out in table 1 above.

In a further specific embodiment, control of a usage session in an aerosol-generating device as illustrated in FIGS. 1 to 4 may be determined with respect to volume of aerosol delivered to a user during the usage session. During a usage session, a user may wish to have an indication of progress through the usage session. For example, the user may wish to know approximately how much potentially deliverable aerosol he has remaining, or approximately how much time there remains in the usage session.

Thus, in a specific embodiment the aerosol-generating device is configured such that each usage session has a duration of 6.5 minutes (390 seconds) from initiation of the usage session, or delivery of a predetermined maximum volume of aerosol, if that predetermined volume of aerosol is delivered to the user within 6.5 minutes from initiation of the usage session. The predetermined maximum volume of aerosol may be, for example, 750 ml of aerosol. The threshold values for the time or aerosol-volume may be set to be any suitable number.

The controller is configured to detect puffs taken during a usage session. A puff start point and a puff end point for each detected puff is determined by monitoring power supplied to the heater during the usage session. When a user takes a puff, the flow of air cools the heater and, therefore, a greater amount of energy is supplied by the battery to maintain the temperature of the heater at its operational temperature. Thus, by monitoring power supplied by the heater, the controller is able to determine the start point and the end point of puffs taken during a usage session. By integrating the monitored power between the detected puff start point and the detected puff end point, a calculated value for aerosol delivered may be obtained. By summing the calculated values of aerosol delivered during the usage session, a cumulative value of aerosol delivered during the usage session may be obtained.

In order to monitor progress, a usage session is split into a number of sequential phases starting with a first phase starting when the usage session starts and ending with a final phase when the usage session ends, passage from one phase to a next phase being determined by time and cumulative volume of aerosol delivered. As the usage session progresses through its sequential phases, the controller instructs the OLED display to emit signals indicative of each successive phase. Thus, a user knows approximately the progress of the usage session.

In the specific example, a usage session controlled according to the second selected criteria may be broken into thirteen sequential phases for indication purposes. The user inserts an aerosol-generating article 30 into the cavity 25 of the device 10 and initiates a usage session by tapping the touch sensitive OLED display 60. The timer is then initiated to record time elapsed during the usage session and the controller is initiated to identify puffs taken during the usage session and calculate volume of aerosol delivered during each of the puffs. A first phase of the usage session is deemed to have started when the usage session started.

While in the first phase the controller instructs the OLED display 60 to emit a signal indicative of the usage session being in the first phase. The first phase ends and the second phase begins after 30 seconds have elapsed from the start of the usage session, or after a first predetermined volume of aerosol has been delivered since the start of the usage session, if the first predetermined volume of aerosol is delivered before 30 seconds has elapsed from the start of the usage session. The first predetermined volume of aerosol may be, for example, 60 ml.

The second phase of the usage session is deemed to have started when the first phase has ended. While in the second phase the controller instructs the OLED display 60 to emit a signal indicative of the usage session being in the second phase. The second phase ends and the third phase begins after 60 seconds have elapsed from the start of the usage session, or after a second predetermined volume of aerosol has been delivered since the start of the usage session, if the second predetermined volume of aerosol is delivered before 60 seconds has elapsed from the start of the usage session. The second predetermined volume of aerosol may be, for example, 120 ml.

This process is repeated for each of the third to thirteenth phases. After the thirteenth and final phase, the usage session ends.

Information regarding the indication state associated with each phase and the criteria for ending each phase is set out in table 4 below.

TABLE 4
Phases of a usage session including displayed indication state using the
second criteria for ending each phase in terms of aerosol volume or time
from the start of the usage session.

| | Displayed | Second Criteria for ending phase | | |
|---|---|---|---|---|
| Phase | Indication | Aerosol | | Time |
| No. | State | Volume (ml) | | (s) |
| 1 | 1 | 60 | or | 30 |
| 2 | 2 | 120 | or | 60 |
| 3 | 3 | 180 | or | 90 |
| 4 | 4 | 240 | or | 120 |
| 5 | 5 | 300 | or | 150 |
| 6 | 6 | 360 | or | 180 |
| 7 | 7 | 420 | or | 210 |
| 8 | 8 | 480 | or | 240 |
| 9 | 9 | 540 | or | 270 |
| 10 | 10 | 600 | or | 300 |
| 11 | 11 | 660 | or | 330 |
| 12 | 12 | 720 | or | 360 |
| 13 | 13 | 750 | or | 390 |

The example above divides a usage session into thirteen sequential phases, each phase ending when the second criteria regarding aerosol volume delivered or time elapsed are fulfilled. Each of the thirteen sequential phases can be represented by one of thirteen sequential indication states. As an example, where the aerosol-generating device has an indicator in the form of an OLED display, the thirteen sequential indication states may be those set out in table 1 above.

An aerosol-generating device may undergo a number of different operational events. It may also be desirable for a user to be able to determine the progress or status of one or more of these events. In a further example an aerosol-generating device is configured to undergo a pre-heating operation to increase the temperature of a heater from an ambient temperature to an operational temperature before the start of a usage session. Such a preheating operation or preheating mode may be part of a usage session, but may also be instigated before a usage session. As described above in relation to usage sessions, a preheating operation may be divided into a number of sequential phases and progress through each one of those sequential phases may be represented by one of a number of indication states.

Progress through a preheating operation may be controlled with respect to temperature. Time may also be a control parameter, although the device may be configured such that it does not operate a usage session if the temperature of the heater does not reach a predetermined operating temperature.

In a specific example using an aerosol-generating device as illustrated in FIGS. 1 to 4, a preheating operation is controlled by temperature of a heater and is divided into thirteen sequential phases, each phase ending when temperature of the heater meets a predetermined threshold. The temperature of the heater may be monitored directly, for example by use of a temperature sensor such as a thermistor or thermocouple. Alternatively, the temperature of the heater may be derived by monitoring other parameters, for example by monitoring current and/or voltage supplied to the heater. When the preheating operation is initiated, power is supplied to the heater and the temperature of the heater increases. In a specific example the preheating phase may end when the temperature of the heater reaches 390° C. The temperature at the end of the preheating phase may be varied to any suitable temperature. It is noted that the temperature at the end of a preheating phase may be higher or lower than a desired operating temperature for generating aerosol during a usage session.

Table 5 below sets out the phases and criteria for determining and displaying progress of a preheating operation.

TABLE 5
Phases of a preheating operation including displayed indication
state and criteria for ending each phase in terms of temperature.

| Phase | Displayed | Criteria for ending phase |
|---|---|---|
| No. | Indication State | Temperature (° C.) |
| 1 | 1 | 30 |
| 2 | 2 | 60 |
| 3 | 3 | 90 |
| 4 | 4 | 120 |
| 5 | 5 | 150 |
| 6 | 6 | 180 |
| 7 | 7 | 210 |
| 8 | 8 | 240 |
| 9 | 9 | 270 |
| 10 | 10 | 300 |
| 11 | 11 | 330 |
| 12 | 12 | 360 |
| 13 | 13 | 390 |

The example above divides a preheating operation into thirteen sequential phases, each phase ending when certain criteria regarding temperature of a heater are fulfilled. Each of the thirteen sequential phases can be represented by one of thirteen sequential indication states. As an example, where the aerosol-generating device has an indicator in the form of an OLED display, the thirteen sequential indication states may be those set out in table 1 above.

In specific embodiments, an aerosol-generating device as illustrated in FIGS. 1 to 4 may be configured to determine progress through and display progress relating to more than one event. As a specific example, an aerosol generating device may be configured to undergo a preheating operation, immediately followed by a usage session. The user inserts an aerosol-generating article 30 into the cavity 25 of the device 10 and initiates a preheating operation by tapping the touch sensitive OLED display. Power is supplied to a heater of the device, and the controller is initiated to determine temperature of the heater. A first phase of the preheating operation is deemed to have started when the preheating operation starts. As the temperature of the heater increases, the controller instructs the visual indicator to display the sequence of indication states shown in table 4 above. The indication states may conveniently display the progress of the preheating operation as an increasing overall luminance provided by the OLED display 60, for example as represented in FIG. 6a to 6d.

The usage session starts as soon as the preheating operation has ended. The usage session may progress, for example, as set out above in table 5. The indication states may conveniently display the progress of the usage session as a waxing ring of luminance, for example as described in relation to FIGS. 5a to 5f and table 1 above.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number “A” is understood as “A”±10% of “A”. Within this context, a number “A” may be considered to include numerical values that are within general standard error for the measurement of the property that the number “A” modifies. The number “A”, in some instances as used in the appended claims, may deviate by the percentages enumerated above provided that the amount by which “A” deviates does not materially affect the basic and novel characteristic(s) of the claimed invention. Also, all ranges include the maximum and minimum points disclosed and include any intermediate ranges therein, which may or may not be specifically enumerated herein.

1.-15. (canceled)

16. An aerosol-generating device for generating an aerosol from an aerosol-forming substrate, the aerosol-generating device comprising an OLED display configured to display data indicative of a state of operation of the aerosol-generating device, wherein the OLED display is controlled to display an outer illumination zone partially or wholly surrounding an inner illumination zone, and wherein the OLED display is further controlled to: i) selectively display in one of the outer and the inner illumination zones to generate a predetermined first display conveying first information, and ii) selectively activate the other of the outer and the inner illumination zones to generate a predetermined second display conveying second information.

17. The aerosol-generating device according to claim 16, further comprising a controller configured to control the OLED display to display data regarding operation of the aerosol-generating device.

18. The aerosol-generating device according to claim 16,
further comprising a housing, at least a portion of the housing having a curved external profile,
wherein the OLED display is an OLED screen conforming to the at least a portion of the housing having the curved external profile.

19. The aerosol-generating device according to claim 16, wherein the OLED display is a user interface.

20. The aerosol-generating device according to claim 19, wherein the OLED display is touch sensitive to enable user input, or is a user input element having a capacitive touch sensitivity.

21. The aerosol-generating device according to claim 16, wherein the aerosol-generating device is configured to detect user interaction with the OLED display, convert the user interaction into a user interaction signal, and use the user interaction signal to control one or more aspects of the aerosol-generating device.

22. The aerosol-generating device according to claim 21,
wherein the detected user interaction is a user touch on the OLED display, and
wherein the user interaction signal is configured to control one or more operational aspects of the aerosol-generating device.

23. The aerosol-generating device according to claim 21, wherein the aerosol-generating device is further configured to recognise and distinguish two or more different user interactions with the OLED display, each of the two or more different user interactions being a different user input.

24. The aerosol-generating device according to claim 23, wherein the two or more user interactions are selected from the list consisting of a single tap, a double tap, a treble tap, a prolonged touch, a swipe left, a swipe right, a swipe up, a swipe down, and a swipe in a pattern.

25. The aerosol-generating device according to claim 16, further comprising a graphic display module configured to drive the OLED display.

26. The aerosol-generating device according to claim 16, further comprising a memory configured to store one or more graphic files defining graphics to be displayed by the OLED display.

27. The aerosol-generating device according to claim 16,
wherein the aerosol-generating device is configured to monitor progress of an operational event,
the aerosol-generating device further comprising a controller configured to control the OLED display to display the progress of the operational event.

28. The aerosol-generating device according to claim 27, wherein the aerosol-generating device is further configured to monitor progress of a usage session during which aerosol is generated and to display the progress of the usage session.

29. The aerosol-generating device according to claim 27, wherein the aerosol-generating device is configured to monitor a parameter relating to progress of the event.

30. The aerosol-generating device according to claim 16, wherein the OLED display is controlled to display progress of an event as a sequence of lighting states.

31. The aerosol-generating device according to claim 30, wherein the event is a usage session.

32. The aerosol-generating device according to claim 16, wherein the outer illumination zone circumscribes at least 50% of a perimeter of the inner illumination zone.

33. The aerosol-generating device according to claim 32, wherein the first information relates to progression of an operational phase of the aerosol-generating device, the second information relates to a state of the aerosol-generating device, the predetermined first display is a predetermined sequence or animation, and the predetermined second display is a predetermined symbol, logo, or light emission.
